(19) United States
(12) Patent Application Publication
(10) Pub. No.: US 2024/0407908 A1
(43) Pub. Date: Dec. 12, 2024

(54) STENT AND METHOD FOR MANUFACTURING SAME

(71) Applicant: S&G BIOTECH, INC., Yongin-si (KR)

(72) Inventors: Sung Kwon KANG, Yongin-si (KR); Keun HER, Incheon (KR); Young Jae LEE, Seongnam-si (KR); Chang Gi MOON, Yongin-si (KR); Gu Hyun LEE, Seongnam-si (KR)

(21) Appl. No.: 18/273,999

(22) PCT Filed: Jan. 27, 2022
(86) PCT No.: PCT/KR2022/001491

§ 371 Date: Jul. 25, 2023

(30) Foreign Application Priority Data
Jan. 27, 2021 (KR) 10-2021-0011666

(51) Int. Cl. A61F 2/07; A61F 2/06

(52) U.S. Cl. CPC A61F 2/07; A61F 2002/065; A61F 2002/075; A61F 2230/006; A61F 2240/001

(57) ABSTRACT

A stent (1) of the present invention comprises: a main body (2) formed in a hollow tube shape; and a first bifurcation (3) and a second bifurcation (4) which are formed by extending from the end portion of the main body (2) in the lengthwise direction of the main body (2). The stent (1) comprises an artificial blood vessel (5) consisting of a main blood vessel (51), and a first branch blood vessel (52) and a second branch blood vessel (53) branched from the main blood vessel (51). The main body (2) is arranged to surround the main blood vessel (51), the first bifurcation (3) is arranged to surround part of the first branch blood vessel (52), and the second bifurcation (4) is arranged to surround part of the second branch blood vessel (53). The stent (1) includes a plurality of cells (10) which are formed by weaving one wire in zigzags, and the cells (10) each include an end cell (20) formed at the end portion of the main body (2). In addition, the first bifurcation (3) and the second bifurcation (4) are formed at the lower end of the end cell (20) as one wire integrally with the main body (2).

### TECHNICAL FIELD

The present invention relates to a stent and a method for preparing the stent, and more particularly, to a stent for preventing a branched portion of a branched stent from being bent and folded and a method for preparing the same.

### BACKGROUND ART

Living bodies contain several passages, including artery, urinary, biliary, tracheobronchial, esophageal, or renal tract. The passages may be obstructed, weakened, or require structural support. For example, the passages may be obstructed by a tumor, restricted by plaque, or weakened by an aneurysm. In this case, a medical prosthesis may be inserted or planted in the passages.

In most cases, the prosthesis is a tubular member and includes, for example, a stent, a stent graft, a covered stent, or an aortic valve. Some of the passages may have a bifurcation structure. For example, the abdominal aorta branches into the left iliac artery and the right iliac artery, and certain cases of abdominal aortic aneurysm require the insertion of a bifurcated stent suitable for this bifurcated passage for treatment. In particular, in a procedure for inserting a branched stent to treat an abdominal aortic aneurysm, there are cases in which guide wires must be inserted into the branched stent several times in clinical practice. In this case, when the branched stent is bent and folded, the guide wire cannot be inserted into the branched stent, and the dejected guide wire causes the branched stent to depart from its original position. In particular, when a patient's blood vessel is curved, the branched stent is more easily folded, and the branched stent has a problem in that it is more easily folded because the branched portion has a reduced diameter.

### DISCLOSURE

### Technical Problem

In view of these circumstances, the present invention is to provide a stent and a method for preparing the stent that can prevent the branched portion of the stent from being folded.

### Technical Solution

A stent according to an embodiment of the present invention for addressing the above issues comprises a main body 2 formed in a hollow tube shape and a first bifurcation 3 and a second bifurcation 4 which are formed by extending from an end of the main body 2 in the longitudinal direction of the main body 2, in which the stent 1 may comprise an artificial blood vessel 5 consisting of a main blood vessel 51, a first branch blood vessel 52 and a second branch blood vessel 53 branched from the main blood vessel 51, the main body 2 may be arranged to surround the main blood vessel 51, the first bifurcation 3 may be arranged to surround a portion of the first branch blood vessel 52, the second bifurcation 4 may be arranged to surround a portion of the second branch blood vessel 53, the stent 1 may include a plurality of cells 10 which are formed by weaving one wire in zigzags, each of the cells 10 may include an end cell 20 formed at the end of the main body 2, and the first bifurcation 3 and the second bifurcation 4 may be formed as one wire integrally with the main body 2 at the lower end of the end cell 20.

Further, in one embodiment, the first bifurcation 3 may be formed by weaving additional cells with a continuous portion of the end cell 20 of the main body 2 in the longitudinal direction of the main body 2, and the second bifurcation 4 may be spaced apart from the first branch bifurcation 3 and formed by weaving additional cells with another continuous portion of the end cell 20 of the main body 2 in the longitudinal direction of the main body 2.

Further, in one embodiment, the stent 1 may comprise a first reinforcing structure 6 comprising a plurality of grid patterns, and each grid pattern may comprise the end cell 20 and a cell of the first bifurcation 3 or the end cell 20 and a cell of the second bifurcation 4.

Further, in one embodiment, the stent 1 may comprise a zigzag-shaped second reinforcing structure 7, the second reinforcing structure 7 may successively passed through the first to fourth vertices 7.1, 7.2, 7.3, 7.4, and the first vertex 7.1 may be in the main body 2, the second vertex 7.2 may be in the first bifurcation 3, the third vertex 7.3 may be in the main body 2, and the fourth vertex 7.4 may be in the second bifurcation 4.

A method for preparing a stent according to an embodiment of the present invention for addressing the above issues, the stent comprising a main body 2 formed in a hollow tube shape; and a first bifurcation 3 and a second bifurcation 4 which are formed by extending from an end of the main body 2 in the longitudinal direction of the main body 2, in which the stent 1 may comprise an artificial blood vessel 5 consisting of a main blood vessel 51, a first branch blood vessel 52 and a second branch blood vessel 53 branched from the main blood vessel 51, the main body 2 may be arranged to surround the main blood vessel 51, the first bifurcation 3 may be arranged to surround a portion of the first branch blood vessel 52, the second bifurcation 4 may be arranged to surround a portion of the second branch blood vessel 53, the stent 1 may include a plurality of cells 10 which are formed by weaving one wire in zigzags, each of the cells 10 may include an end cell 20 formed at the end of the main body 2, and the method may comprise step (a) of forming the first bifurcation 3 as one wire integrally with the main body 2; and step (b) of forming the second bifurcation 4 as one wire integrally with the main body 2.

Further, in one embodiment, step (a) may comprise forming the first bifurcation 3 by weaving additional cells with a continuous portion of the end cell 20 of the main body 2 in the longitudinal direction of the main body 2, and step (b) may comprise forming the second bifurcation 4 spaced apart from the first bifurcation 3 and by weaving additional cells with another continuous portion of the end cell 20 of the main body 2 in the longitudinal direction of the main body 2.

Further, in one embodiment, the method may further comprise step (c) of forming a first reinforcing structure 6 comprising a plurality of grid patterns, and each grid pattern may comprise the end cell 20 and a cell of the first bifurcation 3 or the end cell 20 and a cell of the second bifurcation 4.

Further, in one embodiment, the method may further comprises step (d) of forming a zigzag-shaped second reinforcing structure 7, in which the second reinforcing structure 7 may successively passed through the first to fourth vertices 7.1, 7.2, 7.3, 7.4, the first vertex 7.1 may be in the main body 2, the second vertex 7.2 may be in the first bifurcation 3, the third vertex 7.3 may be in the main body 2, and the fourth vertex 7.4 may be in the second bifurcation 4.

### Advantageous Effects

According to the present invention, it is possible to prevent the stent from being folded due to a break between the main body and the first bifurcation or the second bifurcation.

Further, the first and second bifurcations may be integrally formed as a single wire at the lower ends of the end cells of the main body, thereby improving manufacturing efficiency and manufacturing workability.

### DESCRIPTION OF DRAWINGS

FIG. 1 shows the appearance of the stent as an example of a stent according to the present invention.

FIG. 2 shows the appearance of the stent without the artificial blood vessel in the stent according to the present invention.

FIG. 3 is a development view of the stent according to the present invention.

FIG. 4 is a development view of the first reinforcing structure of the present invention.

FIG. 5 is a development view of the second reinforcing structure of the present invention.

FIG. 6 shows a state in which the stent is additionally attached to the artificial blood vessel 5 in the stent according to the present invention.

FIG. 7 shows the stent according to the present invention viewed from the top.

### MODE FOR INVENTION

Hereinafter, embodiments of the present invention are described with reference to the accompanying drawings. Further, the present invention is not limited to the following embodiments, and may be realized in any arbitrary possible combination as well as in a separate manner recognizable by a person skilled in the art. Further, the scope of the present invention is not limited by the preferred embodiments. Further, embodiments are shown schematically in the drawings. In this application, like reference numbers in these drawings indicate identical or functionally similar components.

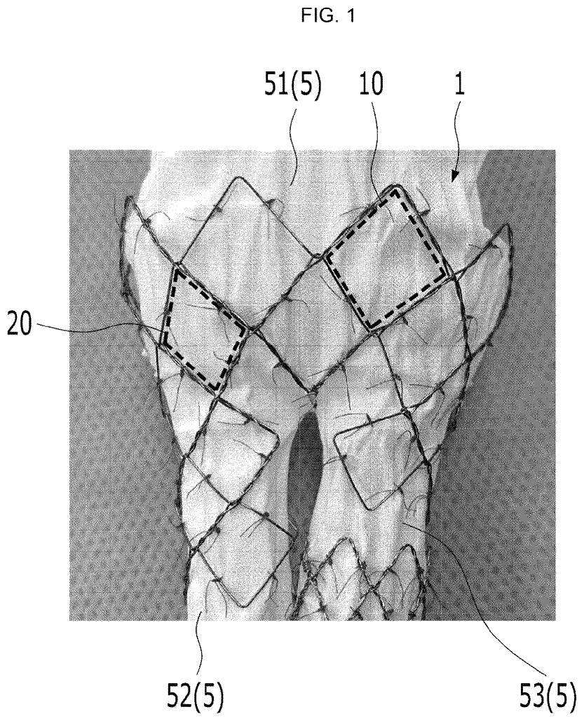

FIG. 1 shows the stent 1 of the present invention.

Referring to FIG. 1, the stent 1 of the present invention comprises the artificial blood vessel 5 consisting of the main blood vessel 51, the first branch blood vessel 52, and the second branch blood vessel 53 branched from the main blood vessel 51.

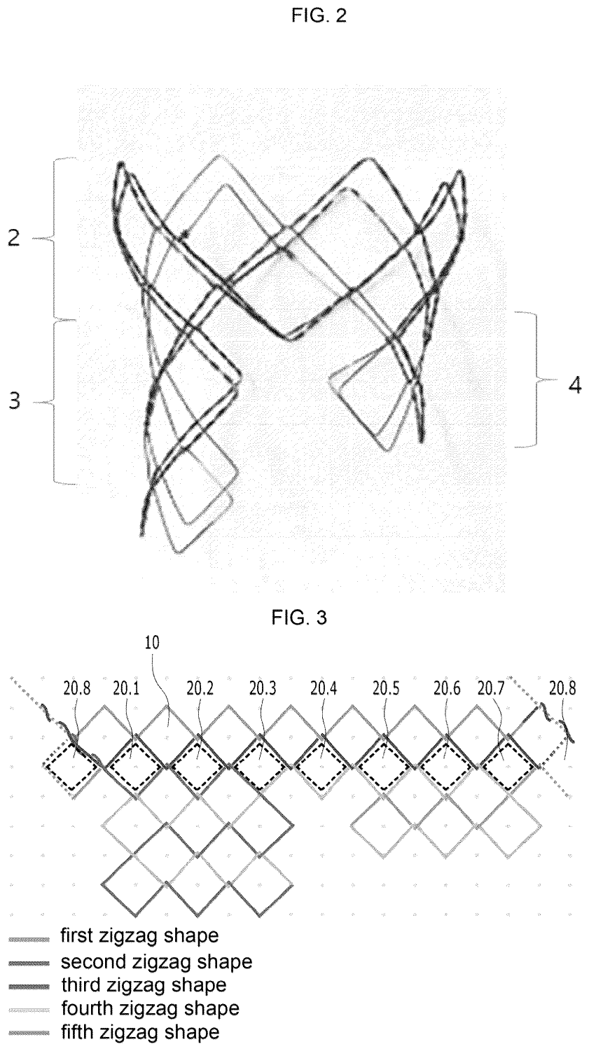

FIG. 2 shows the appearance without the artificial blood vessel 5 in the stent 1 of the present invention.

Referring to FIG. 2, the stent 1 of the present invention comprises the main body 2 formed in a hollow tube shape and the first bifurcation 3 and the second bifurcation 4, which are formed by extending from an end of the main body 2 in the longitudinal direction of the main body 2. Referring to FIG. 1 and FIG. 2, the main body 2 is arranged to surround the main blood vessel 51, the first bifurcation 3 is arranged to surround a portion of the first branch blood vessel 52, and the second bifurcation 4 is arranged to surround a portion of the second branch blood vessel 53.

Further, the stent 1 includes a plurality of cells 10, which are formed by weaving one wire in zigzags, and each of the cells 10 includes the end cell 20 formed at the end of the main body 2. Further, the first bifurcation 3 and the second bifurcation 4 are formed as one wire integrally with the main body 2 at the lower end of the end cell 20. Preferably, the main body 2, the first bifurcation 3, and the second bifurcation 4 are formed of one wire.

Specifically, the first bifurcation 3 is formed by weaving additional cells with a continuous portion of the end cell 20 of the main body 2 in the longitudinal direction of the main body 2, and the second bifurcation 4 is spaced apart from the first bifurcation 3 and formed by weaving additional cells with another continuous portion of the end cell 20 of the main body 2 in the longitudinal direction of the main body 2.

For example, FIG. 3 shows a development view of a case in which the stent 1 according to the present invention is composed of eight end cells 20, that is, first to eighth end cells 20.1, 20.2, 20.3, 20.4, 20.5, 20.6, 20.7, 20.8 in a counterclockwise direction. The first bifurcation 3 extends from the first to third end cells 20.1, 20.2, and 20.3, and the second bifurcation 4 extends from the fifth to seventh end cells 20.5, 20.6, and 20.7.

For this reason, the stent 1 of the present invention provides a branched stent 1, while the force applied to the first bifurcation 3 and the second bifurcation 4 is distributed to the main body 2, thereby preventing folding between the main body 2 and the first bifurcation 3 or between the main body 2 and the second bifurcation 4. Further, it can increase manufacturing efficiency and manufacturing workability.

Meanwhile, according to an embodiment of the present invention, the stent 1 comprises the first reinforcing structure 6 comprising a plurality of grid patterns. Each grid pattern comprises the end cell 20 and a cell of the first bifurcation 3 or the end cell 20 and a cell of the second bifurcation 4.

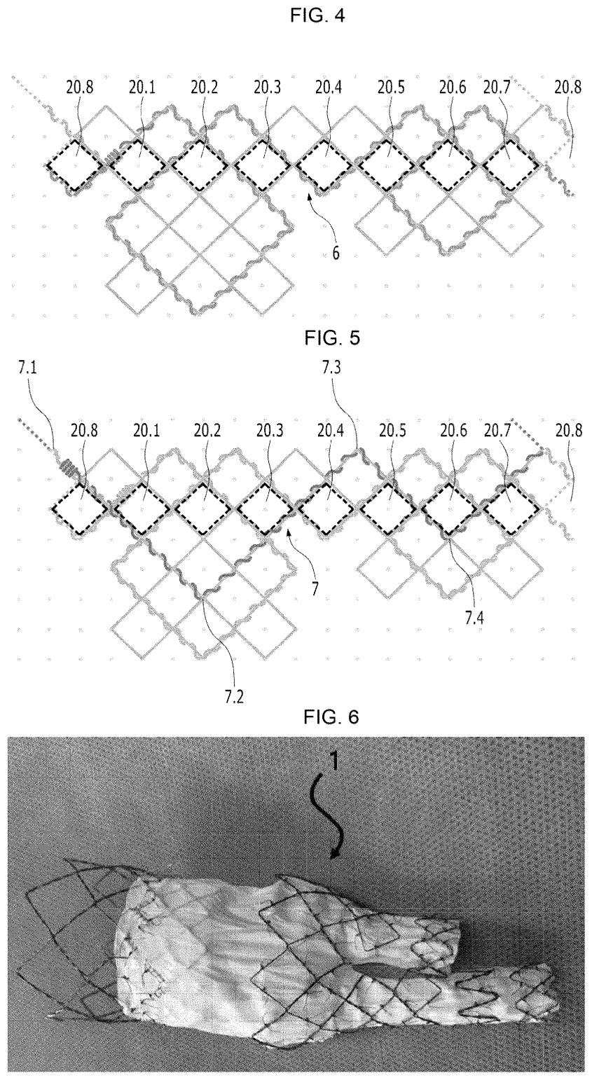

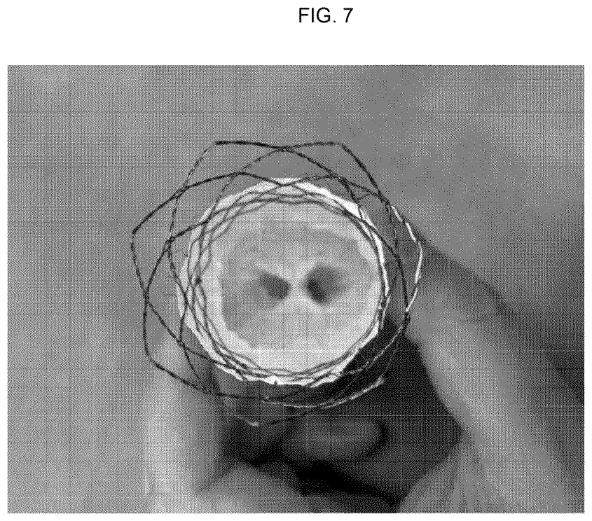

For example, as shown in FIG. 4, the first reinforcing structure 6 forms a grid pattern passing through the first bifurcation 3 by drawing a rectangle with the upper end of the second end cell 20.2 as one vertex. Further, the first reinforcing structure 6 forms a grid pattern passing through the second bifurcation 4 by drawing a rectangle with the upper end of the sixth end cell 20.6 as one vertex. Preferably, the first reinforcing structure 6 is a grid pattern formed in a twisted structure.

For this reason, the stent 1 of the present invention, the first reinforcing structure 6 to reinforce the first bifurcation 3, the second bifurcation 4 and the part of the main body 2 where the first bifurcation 3 and the second bifurcation 4 extend, thereby preventing folding between the main body 2 and the first bifurcation 3 and between the main body 2 and the second bifurcation 4. Further, it can increase manufacturing efficiency and manufacturing workability.

Meanwhile, according to an embodiment of the present invention, the stent 1 comprises the zigzag-shaped second reinforcing structure 7. For example, as shown in FIG. 5, the second reinforcing structure 7 successively passes through the first to fourth vertices 7.1, 7.2, 7.3, 7.4, the first vertex 7.1 is in the main body 2, the second vertex 7.2 is in the first bifurcation 3, the third vertex 7.3 is in the main body 2, and the fourth vertex 7.4 is in the second bifurcation 4. Preferably, the second reinforcing structure 7 is formed in a twisted structure. For this reason, it is possible to more effectively prevent folding between the main body 2 and the first bifurcation 3 and between the main body 2 and the second bifurcation 4.

Next, a method for preparing the stent 1 of the present invention is described with reference to FIGS. 3, 4, and 5.

As shown in FIG. 3, the method for preparing the stent 1 of the present invention, the stent comprising a main body 2 formed in a hollow tube shape; and a first bifurcation 3 and a second bifurcation 4 which are formed by extending from an end of the main body 2 in the longitudinal direction of the main body 2, in which the stent 1 comprises an artificial blood vessel 5 consisting of a main blood vessel 51, a first branch blood vessel 52 and a second branch blood vessel 53 branched from the main blood vessel 51, the main body 2 is arranged to surround the main blood vessel 51, the first bifurcation 3 is arranged to surround a portion of the first branch blood vessel 52, the second bifurcation 4 is arranged to surround a portion of the second branch blood vessel 53, the stent 1 includes a plurality of cells 10 which are formed by weaving one wire in zigzags, each of the cells 10 includes an end cell 20 formed at the end of the main body 2, and the method comprises step (a) of forming the first bifurcation 3 as one wire integrally with the main body 2; and step (b) of forming the second bifurcation 4 as one wire integrally with the main body 2.

Specifically, step (a) comprises forming the first bifurcation 3 by weaving additional cells with a continuous portion of the end cell 20 of the main body 2 in the longitudinal direction of the main body 2, and step (b) comprises forming the second bifurcation 4 spaced apart from the first bifurcation 3 and by weaving additional cells with another continuous portion of the end cell 20 of the main body 2 in the longitudinal direction of the main body 2.

For example, referring to FIG. 3, the first zigzag shape is formed with one wire, and the cell 10 is formed by interweaving the first zigzag shape with the same wire to form the second zigzag shape. Further, step (a) is a process of forming the first bifurcation 3 by weaving the third zigzag shape and the fourth zigzag shape with the same wire as the wire forming the first and second zigzag shapes. Further, step (b) is a process of forming the second bifurcation 4 by weaving the fourth zigzag shape and the fifth zigzag shape with the same wire.

Meanwhile, according to one embodiment of the present invention, the method for preparing the stent 1 further comprises step (c) of forming a first reinforcing structure 6 comprising a plurality of grid patterns, and each grid pattern comprises the end cell 20 and a cell of the first bifurcation 3 or the end cell 20 and a cell of the second bifurcation 4.

For example, referring to FIG. 4, step (c) is a process of forming a grid pattern passing through the first bifurcation 3 by drawing a rectangle with the upper end of the second end cell 20.2 as one vertex. Step (c) is a process of forming, by the first reinforcing structure 6, a grid pattern passing through the second bifurcation 4 by drawing a rectangle with the upper end of the sixth end cell 20.6 as one vertex.

Preferably, step (c) is a process of forming the first reinforcing structure 6 including a plurality of grid patterns formed in a twisted structure. For this reason, the reinforcing force of the first reinforcing structure 6 can be further increased.

Preferably, in step (c), the first reinforcing structure 6 is made of the same wire as one wire forming the main body 2, the first bifurcation 3, and the second bifurcation 4. For this reason, it can increase manufacturing efficiency and manufacturing workability.

Meanwhile, according to an embodiment of the present invention, the method for preparing the stent 1 further comprises step (d) of forming the zigzag-shaped second reinforcing structure 7. The second reinforcing structure 7 successively passes through the first to fourth vertices 7.1, 7.2, 7.3, 7.4, the first vertex 7.1 is in the main body 2, the second vertex 7.2 is in the first bifurcation 3, the third vertex 7.3 is in the main body 2, and the fourth vertex 7.4 is in the second bifurcation 4.

Preferably, step (d) is a process of forming the zigzag-shaped second reinforcing structure 7 formed in a twisted structure. For this reason, the reinforcing force of the second reinforcing structure 7 can be further increased.

1. A stent (1) comprising:
a main body (2) formed in a hollow tube shape; and
a first bifurcation (3) and a second bifurcation (4) which are formed by extending from an end of the main body (2) in the longitudinal direction of the main body (2),
wherein the stent (1) comprises an artificial blood vessel (5) consisting of a main blood vessel (51), and a first branch blood vessel (52) and a second branch blood vessel (53) branched from the main blood vessel (51),
wherein the main body (2) is arranged to surround the main blood vessel (51), the first bifurcation (3) is arranged to surround a portion of the first branch blood vessel (52), and the second bifurcation (4) is arranged to surround a portion of the second branch blood vessel (53),
wherein the stent (1) includes a plurality of cells (10) which are formed by weaving one wire in zigzags,
wherein each of the cells (10) includes an end cell (20) formed at the end of the main body (2), and
wherein the first bifurcation (3) and the second bifurcation (4) are formed as one wire integrally with the main body (2) at the lower end of the end cell (20).

2. The stent (1) of claim 1, wherein the first bifurcation (3) is formed by weaving additional cells with a continuous portion of the end cell (20) of the main body (2) in the longitudinal direction of the main body (2), and
wherein the second bifurcation (4) is spaced apart from the first branch bifurcation (3) and formed by weaving additional cells with another continuous portion of the end cell (20) of the main body (2) in the longitudinal direction of the main body (2).

3. The stent (1) of claim 1, wherein the stent (1) comprises a first reinforcing structure (6) comprising a plurality of grid patterns, and
wherein each grid pattern comprises the end cell (20) and a cell of the first bifurcation (3) or the end cell (20) and a cell of the second bifurcation (4).

4. The stent (1) of claim 3, wherein the stent (1) comprises a zigzag-shaped second reinforcing structure (7),
wherein the second reinforcing structure (7) successively passes through the first to fourth vertices (7.1, 7.2, 7.3, 7.4), and
wherein the first vertex (7.1) is in the main body (2), the second vertex (7.2) is in the first bifurcation (3), the third vertex (7.3) is in the main body (2), and the fourth vertex (7.4) is in the second bifurcation (4).

5. A method for preparing a stent (1) comprising: a main body (2) formed in a hollow tube shape; and a first bifurcation (3) and a second bifurcation (4) which are formed by extending from an end of the main body (2) in the longitudinal direction of the main body (2),
wherein the stent (1) comprises an artificial blood vessel (5) consisting of a main blood vessel (51), and a first branch blood vessel (52) and a second branch blood vessel (53) branched from the main blood vessel (51),
wherein the main body (2) is arranged to surround the main blood vessel (51), the first bifurcation (3) is arranged to surround a portion of the first branch blood vessel (52), and the second bifurcation (4) is arranged to surround a portion of the second branch blood vessel (53),
wherein the stent (1) includes a plurality of cells (10) which are formed by weaving one wire in zigzags,
wherein each of the cells (10) includes an end cell (20) formed at the end of the main body (2), and
wherein the method comprises step (a) of forming the first bifurcation (3) as one wire integrally with the main body (2); and step (b) of forming the second bifurcation (4) as one wire integrally with the main body (2).

6. The method of claim 5, wherein in step (a), forming the first bifurcation (3) by weaving additional cells with a continuous portion of the end cell (20) of the main body (2) in the longitudinal direction of the main body (2), and
wherein in step (b), forming the second bifurcation (4) spaced apart from the first bifurcation (3) and by weaving additional cells with another continuous portion of the end cell (20) of the main body (2) in the longitudinal direction of the main body (2).

7. The method of claim 5, wherein the method further comprises step (c) of forming a first reinforcing structure (6) comprising a plurality of grid patterns, and
wherein each grid pattern comprises the end cell (20) and a cell of the first bifurcation (3) or the end cell (20) and a cell of the second bifurcation (4).

8. The method of claim 7, wherein the method further comprises step (d) of forming a zigzag-shaped second reinforcing structure (7),
wherein the second reinforcing structure (7) successively passes through the first to fourth vertices (7.1, 7.2, 7.3, 7.4), and
wherein the first vertex (7.1) is in the main body (2), the second vertex (7.2) is in the first bifurcation (3), the third vertex (7.3) is in the main body (2), and the fourth vertex (7.4) is in the second bifurcation (4).

9. The stent (1) of claim 2, wherein the stent (1) comprises a first reinforcing structure (6) comprising a plurality of grid patterns, and
wherein each grid pattern comprises the end cell (20) and a cell of the first bifurcation (3) or the end cell (20) and a cell of the second bifurcation (4).

10. The method of claim 6, wherein the method further comprises step (c) of forming a first reinforcing structure (6) comprising a plurality of grid patterns, and
wherein each grid pattern comprises the end cell (20) and a cell of the first bifurcation (3) or the end cell (20) and a cell of the second bifurcation (4).
